(19) United States
(12) Patent Application Publication
(10) Pub. No.: US 2008/0221104 A1
(43) Pub. Date: Sep. 11, 2008

(54) SOLUBLE EPOXIDE HYDROLASE INHIBITORS FOR THE TREATMENT OF RHEUMATOID ARTHRITIS

(75) Inventor: Heather Kay Webb Hsu, San Francisco, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 11/934,642

(22) Filed: Nov. 2, 2007

Related U.S. Application Data
(60) Provisional application No. 60/856,619, filed on Nov. 3, 2006.

(51) Int. Cl. A61K 31/4468; A61K 31/5375; A61P 29/00

(52) U.S. Cl. 514/237.5; 514/329

(57) ABSTRACT

Methods for reducing autoimmune induced inflammation, including rheumatoid arthritis in a subject in need of such therapy are disclosed.

### CROSS-REFERENCE TO RELATED APPLICATION

This application claims the benefit under 35 U.S. C. § 119(e) of U.S. Provisional Patent Application Ser. No. 60/856,619, filed on Nov. 3, 2006, which is incorporated herein by reference in its entirety.

### FIELD OF THE INVENTION

This invention generally relates to a class of urea compounds and related compositions and methods useful for treating and ameliorating the symptoms of autoimmune disorders such as rheumatoid arthritis.

### BACKGROUND

The arachidonate cascade is a ubiquitous lipid signaling cascade that liberates arachidonic acid from the plasma membrane lipid reserves in response to a variety of extra-cellular and/or intra-cellular signals. The released arachidonic acid is then available to act as a substrate for a variety of oxidative enzymes that convert it to signaling lipids that have been implicated in inflammation. Several commercially available drugs target and disrupt this pathway. Non-steroidal anti-inflammatory drugs (NSAIDs) disrupt the conversion of arachidonic acid to prostaglandins by inhibiting cyclooxygenases (COX1 and COX2). Asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs are reported to have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been reported to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102(28):9772-7 (2005)).

Of the inflammatory diseases affected by the cascade of cellular and biochemical events related to regulation of sEH activity, none is as pervasive as rheumatoid arthritis (RA). Rheumatoid arthritis is a chronic systemic autoimmune disease of undetermined etiology involving primarily the synovial membranes and articular structures of multiple joints as well as other organs. RA is often progressive and results in pain, stiffness, and swelling of joints. In late stages of the disease, deformity and ankylosis can develop.

Arthritis has a significant financial and social impact for both individuals and the greater society. Prevalence is estimated at 1% in the United States or approximately 3 million people suffering from RA. (Yurdakel, J. Rheumatol. 33:1710 (2006)).

Proinflammatory cytokines, notably interleukin 1 (IL-1) and tumor necrosis factor-alpha (TNF-α), play an important role in initiating and perpetuating inflammatory and destructive processes in the rheumatoid arthritic joint. These cytokines regulate many nuclear factor kappaB (NF-κB) inducible genes that control expression of other cytokines, cell adhesion molecules, immunoregulatory molecules, and proinflammatory mediators. The expression of cyclooxygenase-2 (COX2) and inducible nitric oxide synthase (iNOS) and thereby production of prostaglandins (PG) and NO are regulated by cytokines. PGE2 and NO further promote inflammation and likely participate in destructive mechanisms in the rheumatoid joint. In some experimental systems, the effects of IL-1 and TNF-α appear synergistic, and correspondingly, concomitant inhibition of both cytokines provides greater than additive anti-arthritic effects. (Henderson and Pettipher, Clin. Exp. Immunol. 75:306-310 (1989)). In subjects with active rheumatoid arthritis, blockade of either cytokine results in clinical improvement and less radiographic progression. (Bingham, J. Rheumatol. Supp. 65:3-9 (2002)). Other cytokines such as IL-6 and GM-CSF and chemokines such as IL-8 are also found at elevated levels in rheumatoid arthritis. Conversely, certain other anti-inflammatory cytokines such as IL-10 and TGFβ and cytokine inhibitors such as IL-Ira and soluble TNF-R are often present, however, not at levels generally sufficient to overcome the effects of the cytokines which promote inflammation. (Feldman et al., Ann. Rev. Immunol. 14:397-440 (1996)).

Factors associated with the onset of RA include infectious triggers, genetic predisposition, and autoimmune response. The stimulation of the immune cascade leading to cytokine production, such as tumor necrosis factor alpha (TNF-α) and interleukin-1, generally involves mediation from autoimmune activation of CD4+ T-cells. The primary targets of inflammation are synovial membranes and articular structures. Other organs are affected as well. Inflammation, proliferation, and degeneration typify synovial membrane involvement. Joint deformities and disability result from the erosion and destruction of synovial membranes and articular surfaces.

The American College of Rheumatology published a guide on the treatment of RA emphasizes Disease Modifying Antirheumatic drugs (DMARDs). (Schooff and Wickersham, Am. Fam. Physician 68:849-850 (2003)). Most of the drugs have severe limitations to their use. The gold standard is low dose methotrexate which is associated with a high incidence of liver damage thereby necessitating concomitant monitoring for evidence of liver destruction. Anti-TNFα therapies require injection and thus are less desirable from the subject perspective. Thus, a need exists for effective anti-inflammtory, anti-arthritic agents that have an impact on pain, inflammation and the prevention of joint destruction. This invention satisifies this need and others in the art and provides related advantages as well.

### SUMMARY OF THE INVENTION

This invention provides compounds and compositions that treat, reduce or ameliorate the symptoms associated with rheumatoid arthristis at the clinical and sub-clinical level. The compounds and compositions also are useful to treat, ameliorate or reduce the symptoms associated with additional autoimmune disorders.

In one aspect, the invention provides methods for treating or ameliorating the symptoms of rheumatoid arthritis in a subject in need thereof by administering an effective amount of a compound or composition of this invention. In another aspect, the invention provides a method for reducing autoimmune induced inflammation in a subject in need thereof by administering to the subject an effective amount of a compound or composition of this invention. In a further aspect, methods are provided for reducing autoimmune-induced cytokine levels in a subject having an autoimmune disorder such as rheumatoid arthritis, by administering to the subject an effective amount of a compound or composition of this invention.

As is apparent to the skilled artisan, the amount and dosing schedule of any therapeutic agent will vary with the disease to be treated, the individual and his or her general health. Thus, within these general parameters, the methods described herein require the administration of an effective amount of a compound or a composition containing this compound, wherein the compound is an autoimmune induced inflammation reducing compound of Formula (I):

R¹NHC(═O)NHR² (I)

wherein:

Q is O or S; and

R¹ and R² are independently selected from the group consisting of substituted alkyl, aryl, substituted aryl, heteroaryl, substituted heteroraryl, cyloalkyl, substituted cycloalkyl, heterocycloalkyl, and substituted heterocycloalkyl.

Also provided are autoimmune induced inflammation reducing compounds of Formula (II) or a pharmaceutically acceptable salt thereof:

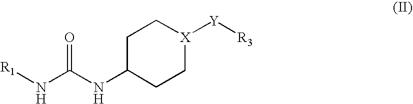

wherein:
R¹ is selected from the group consisting of aryl, substituted aryl, heteroaryl, substituted heteroraryl, cyloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

In a particular aspect of this invention, the compound to be administered is 1-[3-(morpholine-4-carbonyl)-phenyl]-3-(4-trifluoromethyl-phenyl)-urea.

Also provided are methods to identify potential agents for treating or ameliorating the symptoms associated with an autoimmune disorder such as rheumatoid arthritis.

These and other aspects and embodiments of the invention are further described in the text that follows.

### DESCRIPTION OF THE FIGURES

FIG. 1. An arthritis assessment graph is shown comparing degrees of relative inflammation against time for two experimental samples and negative and positive controls as referred to in Table 1.

FIG. 2. A bar graph of plasma levels of IL-6 in mice is shown comparing percent release of IL-6 in response to LPS administration in groups of animals treated with two prospective sEH inhibitors or controls as referred to in Table 1.

FIG. 3. A pharmacokinetic study shows a graph of plasma concentration of sEH inhibitors vs. time. The inhibitors are described in Table 1.

FIG. 4. An arthritis assessment graph is shown comparing degrees of relative inflammation against time for five experimental samples and negative and positive controls as referred to in Table 1.

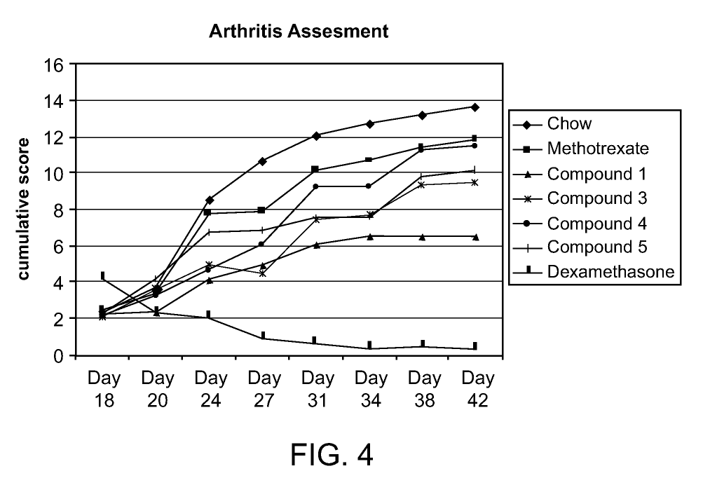

### DETAILED DESCRIPTION THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms have the following defined meanings.

As used in the specification and claims, the singular form “a”, “an” and “the” include plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated, that all numerical designations are preceded by the term “about”. It also is to be understood, although not always explicitly stated, that the reagents described herein are merely exemplary and that equivalents of such are known in the art.

“Cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Alkyl” refers to monovalent saturated aliphatic hydrocarbyl groups having from 1 to 10 carbon atoms and alternatively 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and alternatively 2 to 4 carbon atoms and having at least 1 and alternatively from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and alternatively 2 to 3 carbon atoms and having at least 1 and alternatively from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, alternatively 1 to 3, or more alternatively 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and alternatively 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and alternatively 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, alternatively 1 to 3, or more alternatively 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof “Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

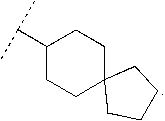

“Cycloalkenyl” refers to non-aromatic cyclic alkyl groups of from 3 to 10 carbon atoms having single or multiple cyclic rings and having at least one >C═C<ring unsaturation and alternatively from 1 to 2 sites of >C═C<ring unsaturation.

“Substituted cycloalkyl” and “substituted cycloalkenyl” refers to a cycloalkyl or cycloalkenyl group having from 1 to 5 or alternatively 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo and alternatively is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, alternatively 1 to 3, or more alternatively 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or alternatively 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

“Stereoisomer” or “stereoisomers” refer to compounds that differ in the chirality at one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or alternatively a human. Mammals include, but are not limited to, murines, rats, simians, humans, farm animals, sport animals and pets.

An “effective amount” is used synonymously with a “therapeutically effective amount” and intends an amount sufficient to effect beneficial or desired results. An effective amount can be administered in one or more administrations, applications, or dosages. It also refers to an amount that will elicit one or more of the following effects: reducing autoimmune-induced inflammation as indicated by reduction in redness, fever, edema, swelling and pain systemically and/or locally, decreasing the levels of inflammatory cytokines and increasing the levels of anti-inflammatory cytokines.

“Inflammation” refers to a localized protective reaction of tissue to irritation, injury, or infection, characterized by pain, redness, swelling, and sometimes loss of function.

“Autoimmune Disease” refers to conditions where inflammation and other conditions associated with malfunction result at least in part by faulty recognition of self by the immune system. Examples of autoimmune diseases include, for example, rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis.

“Rheumatoid Arthritis” or “RA” is a chronic autoimmune disorder characterized by nonspecific, usually symmetric inflammation of the peripheral joints, potentially resulting in progressive destruction of articular and periarticular structures. The ultimate cause can vary but it is generally associated with an autoimmune defect which causes healthy cells to be attacked, triggering the release of factors indicative of inflammation. The “Latex test” is used to diagnose rheumatoid arthritis. This test detects an antibody in the blood called the rheumatoid factor. Rheumatoid factor is not exclusively a product of rheumatoid arthritis, but is a positive indicator for the condition coupled with the pain and inflammation symptoms.

“Treating” or “treatment” of a disease or condition will depend on the disease or condition to be treated and the individual to be treated. In general, treatment intends one or more of (1) inhibiting the progression of the disease or condition as measured by clinical or sub-clinical parameters, (2) arresting the development of the disease as measured by clinical or sub-clinical parameters, (3) ameliorating or causing regression of the disease or condition as measured by clinical or sub-clinical parameters, or (4) reducing pain or discomfort for the subject as measured by clinical parameters.

### Therapeutic Methods

This invention provides compounds and compositions that alleviate, reduce and/or treat symptoms and discomfort associated with an autoimmune disease or condition, e.g., rheumatoid arthritis in a subject in need thereof. As used herein, “a subject in need thereof” intends a subject such as a human patient that presents characteristic symptoms of autoimmune induced inflammation or alternatively has been diagnosed by a health care professional as suffering from such condition.

In one aspect, the subject has elevated levels of an “autoimmune-induced cytokine” which includes any of the cytokines whose increased production in a subject is associated with autoimmune disease, including for example TNF-α, IL-1, IL-6, IL-11, IL-12, IL-17, IL-23, RANTES, MIP-1α, GMCSF, and MCP-1. Methods for determining these levels are known in the art and include, but are not limited to simple, indirect hemagglutination, latex agglutination, ELISA and rate nephelometry. The analysis of an array of cytokines can be performed using a multiplexed cytokine array methodology such as Luminex technology (Clin. Diag. Lab. Immunol. (2003) 10:133-139 and Cytometry B Clin. Cytom. (2004) 1:35-39).

Subjects presenting clinical or sub-clinical evidence of an autoimmune disease are treated by receiving an amount of a compound or composition of this invention that is effective to treat the disease or condition.

Symptoms of autoimmune disease vary widely depending on the type of disease. A group of very nonspecific symptoms often accompany autoimmune diseases especially of the collagen vascular type and include fatigue, dizziness and general malaise. Among the conditions associated with and indicative of a presumptive diagnosis of autoimmune disease are: systemic lupus erythematosus (SLE), Graves' Disease, Hashimoto's Hypothyroidism, celiac disease, gluten intolerance/irritable bowel syndrome, alopecia greata, alopecia totalis, polycystic ovary syndrome (PCOS), Addison's Disease, Cushing's Disease, scleroderma, Sjogren's Syndrome, multiple sclerosis, fibromyalgia, chronic fatigue syndrome and insulin-dependent type 1 diabetes.

Symptoms of autoimmune disease affect essentially all systems of the body. Typically associated with the foregoing diseases are: fever or difficulty maintaining body temperature; hair and scalp abnormalities; skin problems including pigment abnormalities, rashes, itching and sun sensitivity; eye and vision abnormalities, head, neck, throat and vocal disfunction and pain; fatigue and sleep disorders; bone, muscle, tendon and joint swelling and weakness; metabolic changes resulting in weight gain or loss; gastrointestinal symptoms, blood pressure elevation or depression; nervous system disorders such as tremor, numbness, dizziness, vertigo and mental acuity difficulty; gynecological symptoms, infertility and reduced libido.

Tests normally used to screen for autoimmune disease again depend on the presentation of the subject's particular complaint but may include assays for C-reactive protein and screening for anemia. Since the commonality of all autoimmune disorders involves the hyperproliferation of humoral and cell-mediated factors without the concommitant appearance of a causitive pathogen, an infectious agent or allergic history needs to be ruled out before a definitive diagnosis of autoimmune disease is made.

In one aspect, autoimmune induced inflammation is reduced in the subject by administration of an effective amount of the compound or composition described below that will reduce autoimmune induced inflammation. In another aspect, a compound of this invention is co-administered with an effective amount of another active agent such as an inhibitor of sEH.

In one aspect, the methods of this invention treat or reduce inflammation that is associated with a diagnosis of one or more of rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis.

The amount, dosing schedule and route of administration of compound can be determined by the treating physician and will vary with the active agent, and its pharmacological properties, condition to be treated, the severity of the condition, the overall general health of the subject, age, weight and sex of the subject. It should be understood that an effective amount to achieve the desired response is administered.

One can determine if the treatment has been effective for its defined purpose by noting one or more clinical symptoms such as a reduction in pain, redness, swelling and loss of mobility or function. Administration of a compound or compositions of the formulae described below can be further selected on their ability to reduce clinical symptoms by at least 50%, or alternatively, at least by about 60% or alternatively by at least about 70%, or alternatively by at least about 75%, or alternatively by at least about 80%, or alternatively by at least about 85%, or alternatively by at least about 90%, or alternatively by at least about 95%, of pre-administration levels in the subject. When rheumatoid arthritis is treated, reduction of inflammation can be localized to the joints and synovial membranes.

In another aspect, the methods and compositions modulate autoimmune induced cytokine levels (reduced or increased) from pre-administration levels by administering an effective amount of a compound or composition of this invention. In another aspect, the compound or composition is co-administered with an effective amount of another active agent such as an inhibitor of sEH.

Examples of such cytokines include, but are not limited to, TNF-α, IL-1, IL-6, IL-11, IL-12, IL-17, IL-23, RANTES, MIP-1α, GMCSF, and MCP-1. For certain patients, administration of effective amounts of the compounds or compositions increase the proportion of anti-inflammatory cytokine levels from pre-administration levels. Examples of such cytokines include, but are not limited to IL-10 and IL-13.

In one aspect, abnormal cytokine levels of the subject are associated with a diagnosis of one or more of rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis. In a separate aspect, abnormal cytokine levels are associated with a diagnosis of rheumatoid arthritis.

The amount, dosing schedule and route of administration of compound can be determined by the treating physician and will vary with the active agent, and its pharmacological properties, condition to be treated, the severity of the condition, the overall general health of the subject, age, weight and sex of the subject. It should be understood that an effective amount to achieve the desired response is administered.

In certain embodiments, administration of an effective amount of the compound will alter or modulate cytokine levels by at least about 50%, or alternatively at least about 60%, or alternatively by about 70%, or alternatively by about 75%, alternatively by about 80%, alternatively to about 85%, alternatively by about 90% and alternatively by 95%, of cytokine levels as compared to pre-treatment levels.

Examples of such cytokines to be reduced by the methods include, but are not limited to, TNF-α, IL-1, IL-6, IL-11, IL-12, IL-17, IL-23, RANTES, MIP-1α, GMCSF, and MCP-1. For certain patients, an effective amount of a compound or composition is administered to increase the level of IL-10, IL-4 and IL-13, as compared to pre-administration levels. Levels of these cytokines have been reported to be inversely associated with active inflammation (Bohoun, Ann. Pharm. Fr. 59:191-197 (2001)).

Accordingly, in another aspect, the invention provides a method of modulating (increasing or decreasing) the proportion of these cytokines by at least about 50%, or alternatively by at least about 60%, by at least about 70%, by at least about 75%, alternatively by about 80%, alternatively to about 85%, alternatively by about 90% and alternatively by 95%, as compared to pre-treatment levels.

In one aspect, abnormal cytokine levels of the subject are associated with a diagnosis of one or more of rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis. In a separate aspect, abnormal cytokine levels are associated with a diagnosis of rheumatoid arthritis.

In another embodiment of the invention, the inflammatory cytokines are modulated by reducing the level of autoimmune induced cytokines selected from TNF-α, IL-1, IL-6, IL-11, IL-12, IL-17, IL-23, RANTES, MIP-1α, GMCSF, and MCP-1, and concomitantly yet independently increasing the level of anti-inflammatory cytokines selected from IL-10, IL-14 and IL-13, by at least about 50%, or alternatively by at least about 60%, by at least about 70%, by at least about 75%, alternatively by about 80%, alternatively by about 82%, alternatively to about 85%, alternatively by about 90% and alternatively by 95%, as compared to pre-treatment levels.

Also provided are methods to treat or ameliorate the symptoms associated with rheumatoid arthritis by administering an effective amount of a compound described below to the subject to treat or ameliorate said symptoms. The amount, dosing schedule and route of administration of compound can be determined by the treating physician and will vary with the active agent, and its pharmacological properties, condition to be treated, the severity of the condition, the overall general health of the subject, age, weight and sex of the subject. The compounds and compositions can be co-administered with other known or yet to be discovered therapeutic regimens.

In one aspect, the compounds of the formulae provided below and for use in the methods described above also are characterized in presenting the ability, in a comparatively low concentration, to inhibit conversion of a substrate in an in vitro assay. While not intending to be bound by theory, it is well known that the effectiveness of a given enzyme inhibitor for preventing catalysis of the conversion of a substrate to a product is related to the concentration of the inhibitor. Thus, the concentration of the sEH inhibitor required to inhibit sEH activity by 50% is termed the IC₅₀, and it provides a measure of the therapeutic efficacy of a given compound. In an embodiment of the present invention, one or more of autoimmune induced inflammation, modulation of cytokine levels and/or treatment of any associated disease, as described above, is reduced in a subject in need thereof by administering a compound of a formulae defined below, wherein the compound has an IC₅₀ typically of less than about 25 nM and alternatively less than about 10 nM. The technical procedures for determining IC₅₀s for the compounds of the present invention are known in the art as well as being detailed in the Examples.

While the anti-hypertensive effects of other urea derivatives have been recognized, compounds of this invention and others in this class of EETs have not been administered to treat autoimmune disorders and associated inflammation because it was thought endogenous sEH would hydrolyze the EETs too quickly for them to have any useful effect. Surprisingly, it was found during the course of the studies underlying the present invention that exogenously administered inhibitors of sEH succeeded in inhibiting sEH sufficiently that levels of EETs could be further raised by the administration of exogenous EETs. These findings underlie the co-administration of sEH inhibitors and of EETs described above with respect to inhibiting the development and progression of rheumatoid arthritis. This is an important improvement in augmenting treatment. While levels of endogenous EETs are expected to rise with the inhibition of sEH activity caused by the action of the sEH inhibitor, and therefore to result in at least some improvement in symptoms or pathology, it may not be sufficient in all cases to inhibit progression of rheumatoid arthritis fully or to the extent intended. This is particularly true where the diseases or other factors have reduced the endogenous concentrations of EETs below those normally present in healthy individuals. Administration of exogenous EETs in conjunction with an sEH inhibitor is therefore expected to be beneficial and to augment the effects of the sEH inhibitor in reducing the progression of rheumatoid arthritis.

Also provided is a medicament comprising a compound or composition as described herein for use in treating a disease or disorder as described above, which can be identified by noting any one or more clinical or sub-clinical parameters.

Further provided is a method to identify new potential therapeutic agents by comparing the therapeutic effect and pharmacology of the putative therapeutic against the therapeutic benefit and pharmacology of compounds described below. In one aspect, the therapeutic agent is administered to an appropriate animal model, in varying doses and dosing regimens, and its effect to treat or reduce inflammation is determined by any one or more parameters as identified above. Previous to or after this in vivo assay, the ability of the putative therapeutic to inhibit sEH activity can be determined by contacting sEH in vitro with the putative therapeutic and determining the IC₅₀ of the putative therapeutic agent. The IC₅₀ of the compound and its score in the in vivo animal model are compared to a compound described herein. Those having the same or similar scores, alone or in combination, can be selected for further research and development or for use in other assays and methods.

### Compounds for Use in the Therapeutic Methods

In each of the above embodiments, an effective amount of a compound, or composition containing a compound, is administered to a subject in need thereof. The compounds are described by at least one of the following general or specific formula.

In one aspect, the compound is a member of the group of Formula (I):

R¹NHC(═O)NHR² (I)

wherein:
Q is O or S; and
R¹ and R² are independently selected from the group consisting of substituted alkyl, aryl, substituted aryl, heteroaryl, substituted heteroraryl, cyloalkyl, substituted cycloalkyl, heterocycloalkyl, and substituted heterocycloalkyl.

In some aspects, Q is O.

In one aspect, the compound is a member of the group of Formula (II):

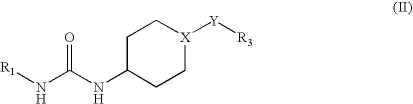

wherein:
R¹ is selected from the group consisting of substituted aryl, substituted aryl, heteroaryl, substituted heteroraryl, cyloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

In some aspects of Formula (I) or (II), R¹ is phenyl optionally substituted with one to three groups independently selected from halo, alkyl, acyl, acyloxy, carboxyl ester, acylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, and alkylsulfonyl.

In some aspects, R¹ is 4-trifluorophenyl.

In some aspects, R¹ is cycloalkyl. In some such aspects, R¹ is adamantyl.

In other aspects, R³ is alkyl. In some such aspects, R³ is methyl.

In other aspects, R³ is heterocycloalkyl. In some such aspects, R³ is morpholino.

In another embodiment, the compound to be administered is a compound, stereoisomer, or a pharmaceutically acceptable salt thereof a compound selected from Table 1.

TABLE 1

| Compound | |
|---|---|
| No. | NAME |
| 1 | 1-[3-(morpholine-4-carbonyl)-phenyl]-3-(4-trifluoromethyl- |
| | phenyl)-urea |
| 2 | 1-(1-acetylpiperidn-4-yl)-3-(adamant-1-yl) urea |
| 3 | 1-(1-acetylpiperidyn-4yl)-3-[4-(trifluoromethyl)phenyl] urea |
| 4 | 1-[1-(methylsulfonyl)piperidin-4-yl]-3-[4- |
| | (trifluoromethyl)phenyl] urea |
| 5 | 1-[1-(methylsulfonyl)piperidin-4-yl]-3-(adamant-1-yl) urea |

### Compositions and Formulations

The compositions are comprised of, in general, a compound of the invention in combination with at least one pharmaceutically acceptable carrier or excipient. Acceptable carriers are known in the art and described supra. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The compounds can be administered in any suitable formulation such as a tablet, pill, capsule, semisolid, gel, transdermal patch or solution, powders, sustained release formulation, solution, suspension, elixir or aerosol. The most suitable formulation will be determined by the disease or disorder to be treated and the individual to be treated.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a compound of the present invention.

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| Compound of the invention | 200 |
| Lactose, spray-dried | 148 |
| Magnesium stearate | 2 |

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Injectable Formulation

The following ingredients are mixed to form an injectable formulation.

| Ingredient | Quantity per injection, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per suppository, mg |
|---|---|
| Compound of the invention | 500 mg |
| Witepsol ® H-15 | balance |

### Combination Therapy

For more generalized therapeutic purposes, wherein the inflammatory condition is present with a syndrome of accompanying illnesses, combination therapy is often desirable. Combination therapy includes administration of a single pharmaceutical dosage formulation which contains a compound of this invention and one or more additional active agents, or therapies such as heat, light and such, as well as administration of the compound and each active agent in its own separate pharmaceutical dosage formulation. For example, a compound of this invention and one or more of other agents such as non-steroidal anti-inflammatory agents (NSAIDs), corticosteroids and disease-modifying antirheumatic drugs (DMARDs), and the like; can be administered to the human subject together in a single oral dosage composition, such as a tablet or capsule, or each agent can be administered in separate oral dosage formulations. Where separate dosage formulations are used, the compound and one or more additional active agents can be administered at essentially the same time (i.e., concurrently), or at separately staggered times (i.e., sequentially). Combination therapy is understood to include all these regimens. Representative DMARDs which may be combined with the sEH inhibitors of the invention include but are not limited to monoclonal antibodies, (infliximab, etanercept or adalimumab), IL-1 antagonists, (anakinra), TNF antagonists, leflunomide, antimalarials, gold salts, sulfasalazine, D-penicillamine, cyclosporin-A, cyclophosphamide and azathiopine.

Complimentary and synergistic compounds can be selected by ability to increase the levels of EETs. This permits EETs to be used in conjunction with one or more sEH inhibitors as described herein to reduce inflammation in the methods of the invention. It further permits EETs to be used in conjunction with one or more sEH inhibitors to reduce clinical and sub-clinical effects described herein. Thus, medicaments of EETs can be made which can be administered in conjunction with one or more sEH inhibitors, or a medicament containing one or more sEH inhibitors can optionally contain one or more EETs.

### Dosing and Administration

The present invention provides therapeutic methods generally involving administering to a subject in need thereof an effective amount of a compound described herein. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The compositions and compounds of the invention and the pharmaceutically acceptable salts thereof are administered via oral, parenteral, subcutaneous, intramuscular, intravenous or topical routes. Generally, the compounds are administered in dosages ranging from about 2 mg up to about 2000 mg per day, although variations will necessarily occur, depending, as noted above, on the target tissue, the subject, and the route of administration. Dosages are administered orally in the range of about 0.05 mg/kg to about 20 mg/kg, more alternatively in the range of about 0.05 mg/kg to about 0.2 mg/kg of body weight per day. The dosage for topical administration will necessarily depend on the size of the area being treated, the disorder to be treated and the individual being treated.

When combined with administration of an effective amount of an EET, the EET can be administered concurrently with the compound, or following administration of or prior to the compounds or vice versa. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Alternatively, the EET or EETs are administered within 24 hours of the inhibitor, and even more alternatively within 12 hours. In increasing order of desirability, the EET or EETs are administered within 10, 8, 6, 4, 2, hours, 1 hour, or one half hour after administration of the inhibitor. The EET or EETs can be are administered concurrently with the inhibitor.

In one embodiment, the EETs, the compounds of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

The following examples are provided to illustrate certain aspects of the present invention and to aid those of skill in the art in practicing the invention. These examples are in no way to be considered to limit the scope of the invention.

### EXPERIMENTAL EXAMPLES

### Synthetic Chemistry

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

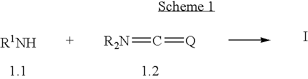

A synthesis of the compounds of the invention is shown in Scheme 1, where Q, R¹, and R² are as previously defined. Amine 1.1 reacts with the appropriate isocyanate 1.2 to form the corresponding urea or thiourea of formula I. Typically, the formation of the urea is conducted using a polar solvent such as DMF (dimethylformamide) at 0 to 10° C. Isocyanate or thioisocyanate 1.2 can be either known compounds or compounds that can be prepared from known compounds by conventional synthetic procedures and include: adamantly isocyanate, cyclohexyl isocyanate, phenyl isocyanate, trifluoromethylphenyl isocyanate, chlorophenyl isocyanate, fluorophenyl isocyanate, trifluoromethoxyphenyl isocyanate.

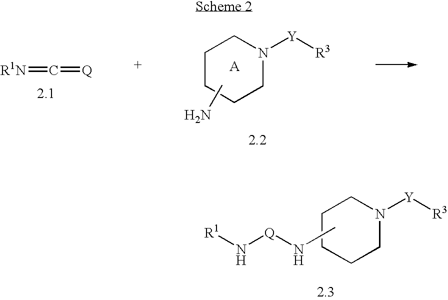

Similarly, compounds of formula (II) may be prepared as shown in Scheme 2 where, for illustrative purposes, ring A is a piperidinyl ring and Q, Y, R¹, and R³ are previously defined. Reaction of isocyanate 2.1 with amine 2.2 forms the corresponding urea or thiourea of 2.3.

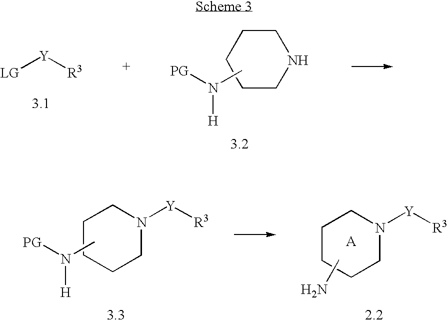

Amine 2.2 can be prepared according to Scheme 3, where LG represents a suitable leaving group such as a halide and PG is an amine protecting group such as a tert-butoxycarbonyl (Boc) group. Reaction of the appropriate isocyanate 3.1 with protected aminopiperidine 3.2 forms the functionalized amine 3.3. Removal of the protecting group gives 2.2.

The following examples are provided to illustrate certain aspects of the present invention and to aid those of skill in the art in practicing the invention. These examples are in no way to be considered to limit the scope of the invention.

### Examples

The examples below as well as throughout the application, the following abbreviations have the following meanings. If not defined, the terms have their generally accepted meanings.
aq.=aqueous
bd=broad doublet
bm=broad multiplet
brs=broad singlet
bt=broad triplet
Boc=tert-Butoxycarbonyl
d=doublet
DCM=dichloromethane
DMAP=dimethylaminopyridine
DMF=dimethylformamide
DMSO=dimethylsulfoxide
eq.=equivalents
EtOAc=ethyl acetate
g=gram
HPLC=high performance liquid chromatography
LCMS=liquid chromatography mass spectroscopy
m=multiplet
M=molar
mg=milligram
MHz=megahertz
mL=milliliter
mM=millimolar
mmol=millimole
m.p.=melting point
MS=mass spectroscopy
N=normal
NMR=nuclear magnetic resonance
s=singlet
t=triplet
TLC=thin layer chromatography
μL=microliters

### Example 1

In all cases in the following examples the designation “Compound X”, where X is a number from 1 to 5, refers to the compounds as identified in Table 1 above.

### 1-(1-Acetyl-piperidin-4-yl)-3-(4-trifluoromethyl-phenyl)-urea

### Preparation of tert-butyl-4-aminopiperidine-1-carboxylate

4-Aminopiperidine (5.0 g, 50 mmol, 1 eq.) was added to a solution of benzaldehyde (5.1 mL, 50 mmol, 1 eq.) in toluene (130 mL) in a 250 mL 3-necked flask fitted with a Dean-Stark trap and a condenser. A nitrogen line was connected to the top of the condenser, and the reaction was refluxed for 3 hours, during which time, water was seen to condense in the Dean-Stark trap. The reaction was cooled to room temperature and Boc anhydride (5.8 mL, 50 mmol, 1 eq.) was added over 5 minutes. The reaction was stirred over night under a blanket of N₂. The solvent was then removed under vacuum and NaHSO₄ (1M in water, 50 mL) was added to the residue. The resulting mixture was stirred vigorously for 2 hours before partitioned between diethyl ether (250 mL) and water (250 mL). The aqueous layer was separated, washed with diethyl ether (3×150 mL) and basified with NaOH solution until the pH was approximately 11. The resulting solution was extracted with DCM (4×200 mL). The combined organic layer was dried over Na₂SO₄, filtered, and evaporated to give 8.0 g of tert-butyl 4-aminopiperidine-1-carboxylate as a yellow oil.

### Preparation of tert-butyl 4-(3-(4-(trifluoromethyl)phenyl)ureido)piperidine-1-carboxylate

4-trifluoromethylphenyl isocyanate (1.0 eq.) was added to a solution of tert-butyl 4-aminopiperidine-1-carboxylate (1 eq.) in ethanol (10 volumes). The reaction mixture was stirred overnight at 50° C. The solvent was removed under vacuum and the crude product was crystallized in diethyl ether to give tert-butyl 4-(3-(4-(trifluoromethyl)phenyl)ureido)piperidine-1-carboxylate as a white solid.

### Preparation of 1-(piperidin-4-yl)-3-(4-(trifluoromethyl)phenyl)urea

tert-Butyl 4-(3-(4-(trifluoromethyl)phenyl)ureido)piperidine-1-carboxylate was stirred in MeOH/HCl overnight. The solvent was removed and the residue was stirred in diethyl ether until a white solid precipitate was seen. The precipitate was collected by filtration to give 1-(piperidin-4-yl)-3-(4-(trifluoromethyl)phenyl)urea as the hydrochloride salt.

### Preparation of 1-(1-acetyl-piperidin-4-yl)-3-(4-trifluoromethyl-phenyl)-urea

### Compound 3

To a solution of 1-(piperidin-4-yl)-3-(4-(trifluoromethyl)phenyl)urea (10.3 g, 35.8 mmol) in DCM (150 mL) cooled with an ice water bath was added sequentially Et₃N (14.9 mL, 107 mmol) and acetic anhydride (5.0 mL, 53.8 mmol). After stirring at room temperature for 18 hours, the resulting precipitate was filtered, washed with DCM (2×50 mL), dried under a high vacuum for 4 hours to give 1-(1-acetyl-piperidin-4-yl)-3-(4-trifluoromethyl-phenyl)-urea as a white solid (8.4 g, 71%). HPLC purity 99.0%; m.p.: 240-248° C.; MS: 330 [M+H]⁺; ¹H NMR (300 MHz, DMSO-d₆) δ: 8.79 (s, 1H, NH), 7.62-7.48 (m, 4H), 6.18 (d, 1H, J=7.5 Hz, NH), 4.11 (d, J=15 Hz, 1H), 3.89-3.72 (m, 2H), 3.08 (t, 1H), 2.91 (m, 1H), 1.99 (s, 3H), 1.85-1.77 (m, 2H), 1.45-1.07 (m, 2H).

### Example 2

### 1-(1-Methanesulfonyl-piperidin-4-yl)-3-(4-trifluoromethyl-phenyl)-urea

### Compound 4

To a solution of 1-(piperidin-4-yl)-3-(4-(trifluoromethyl)phenyl)urea (10.8 g, 37.6 mmol) in DCM (150 mL) cooled with an ice water bath was added sequentially Et₃N (15.7 mL, 113 mmol) and methanesulfonyl chloride (4.37 mL, 56.4 mmol). The reaction was stirred at room temperature for 18 hours. Water (200 mL) was added and the mixture was stirred for another 18 hours. The resulting precipitate was collected by filtration, washed with water (2×50 mL), and dried for 18 hours to give the titled product (3.6 g). The supernatant from the filtration was phase separated. The organic layer was dried over Na₂SO₄, filtered, and concentrated to give an additional 4.0 g of product. The combined crude product (7.6 g) was recrystallized from EtOAc to give the pure product as a white solid (3.15 g, 23%). HPLC purity 93.8%; MS: 366 [M+H]⁺; ¹H NMR (300 MHz, CDCl₃+DMSO-d₆): δ 8.03 (s, 1H, NH), 7.12-7.00 (m, 4H), 5.86 (s, 1H), 3.37-3.20 (m, 3H), 2.95-2.82 (m, 1H), 2.58-2.41 (m, 4H), 1.72-1.58 (m, 2H), 1.24-1.08 (m, 2H).

### Example 3

### 1-[3-(Morpholine-4-carbonyl)-phenyl]-3-(4-trifluoromethyl-phenyl)-urea

### Compound 1

A solution of 4-trifluoromethylphenyl isocyanate (350 mg, 1.87 mmol) and 3-aminocyclohexane carboxylic acid (450 mg, 3.28 mmol) in DMF (10 mL) was warmed at 70° C. overnight. The reaction was monitored by TLC. The reaction mixture was cooled to room temperature and water (5 mL) and 1 N aq. HCl (5 mL) was added with ice bath cooling and stirred for 1 hour. The resulting solid was filtered, washed with water, hexane and dried in a vacuum oven. The crude product was recrystallized from acetone/hexane to afford 310 mg (51%) of product as a white solid. m.p. 271-274.

To a solution of compound (4.3) (254 mg, 0.782 mmol), morpholine (150 mg, 1.72 mmol), and DMAP (102 mg, 0.831 mmol in DCM (15 mL) was added N-[(dimethylamino)propyl]-N′-ethylcarbodiimide hydrochloride (190 mg, 0.991 mmol) at room temperature. The reaction mixture was stirred overnight. The reaction mixture was concentrated and the residue was dissolved in ethyl acetate and washed with 1 N aq. NaOH, 1 N aq. HCl, and water. The ethyl acetate layer was dried over sodium sulphate and concentrated to give the crude product, which was chromatographed on silica gel using EtOAc/MeOH to afford 138 mg (45%) of the product as a white solid. m.p.: 167-171; Mass 394[M+1], ¹HNMR (300 MHz; CDCl₃); δ: 3.5-3.9 (m, 8H, 4*CH₂,); 6.94-7.5 (m, 8H, Ar.CH); 7.8 & 8.2 (brs, 2H, 2*NH); LCMS purity: 98%.

### IC₅₀ Determination

The IC₅₀s for each inhibitor were determined according to the following procedure:

The substrate for the reaction was:

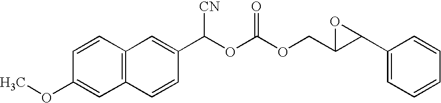

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75).

A standard 96 well plate has rows typically identified by letter and columns identified by numbers. Therefore, well A2 would refer to a well in the first row and second column of the plate.

In a black 96 well plate, all the wells are filled with 150 μL of buffer A (Buffer A: Bis/Tris HCl, 25 mM, pH 7.0 plus 0.1 mg/mL BSA). DMSO (2 microliters) was added in well A2 and A3, and then was added 2 μL of inhibitor solution in A1 and A4 through A12. 150 μL of buffer A was added to row A, then mixed several times and 150 μL of the solution was transferred to row B. This mixing and transfer was repeated up to row H. 20 μL of buffer A was added in column 1 and 2, then 20 μL of enzyme solution was added to columns 3 through 12. The plate was incubated for 5 minutes in the plate reader at 30° C. During incubation, the working solution of substrate was prepared by mixing 3.68 mL of buffer A with 266 μL of 0.5 mM substrate solution. At t=0, 30 μL of working substrate solution was added and readings were started ([S]_{final}: 5 μM). The readings were done at ex: 330 nm (bandwidth 20 nm) and em: 465 nm (bandwidth 20 nm) every 30 second for ten minutes using a fluorescent plate reader (Spectromax M5, Molecular Devices). The velocities were used to analyze and calculate the IC₅₀s. Table 2 shows percent inhibition of Compounds I-5 (as referred to in Table 1) when tested at 50 nM.

TABLE 2

| Compound | % Inhibition at 50 nM |
|---|---|
| 1 | 82 |
| 2 | 89 |
| 3 | 81 |
| 4 | 85 |
| 5 | 94 |

### Bioassays of TNF-α, IL-6 and Rheumatoid Factor

Release of both TNF-α and IL-6 into the bloodstream of animals is associated with events of inflammation. The inflammatory response in turn is associated with a number of predisposing conditions including but not limited to autoimmune and infectious disease. Bacterial endotoxin components of lipopolysaccharide (LPS) are imputed as the primary trigger in septic responses to infection by gram-negative bacteria. Animal models for testing responsiveness to therapeutic agents assay the release of cytokines in response to exposure to LPS. Levels of cytokines before and after treatment are indicators of the effectiveness of, for example, new antibiotic compounds (Prins et al., Infect. Immun. (1995) 63:2236-2242.).

To determine the level of effectiveness of treatment of subjects exhibiting inflammatory disease conditions, the methods of the invention employ standard assays for detection and assessment of released cytokines. By way of illustration only, methods for the blood or serum assay of TNF-α and IL-6 are described herein. Those of skill in the art will recognize that similar methods will apply to the assay procedures for other cytokines, thus, the following description is not intended to be limiting.

For assaying TNF-α, (Engelberts et al., Lymphokine Cytokine Res. (1991) 10:69-76) anti-TNF-α antibody, directed to the 26 kd or 17 kd forms of TNF-α are immobilized and then incubated with the serum or blood sample containing unknown concentrations TNF-α. (Engelberts et al., Lymphokine Cytokine Res. (1991) 10:69-76). After allowing for a suitable period of incubation for antigen-antibody complexes to form, the immobilized antibody is incubated with an indicator solution containing a second anti-TNF-α antibody, either monoclonal or polyclonal, which has been labeled. This second anti-TNF-α antibody is allowed to incubate with immobilized antibody for a sufficient period of time to allow antigen-antibody complexes to form between the labeled antibody and any TNF-α bound by the immobilized monoclonal antibody. After this incubation, the immobilized monoclonal antibody is separated from any unbound labeled antibody, and the amount of label remaining bound to the immobilized antibody is measured. This can be done by measuring a calorimetric or spectrophotometric signal related to the amount of label present on the immobilized antibody. The signal, therefore, provides a measure of the amount of TNF-α in the fluid test sample.

Similarly, IL-6 is assayed most commonly by ELISA, such as provided in commercially available kits, such as the Quantikine IL-6 assay kit (R&D Systems, Minneapolis, Minn.). Briefly, a monoclonal antibody specific for IL-6 coated onto a microtiter plate is used to capture any IL-6 contained in each sample. After washing, an enzyme linked polyclonal antibody specific for IL-6 is added to allow detection of any bound IL-6. Optical density values of samples are recorded (using, for example, a microtiter plate reader from Hewlett Packard) and compared to those of an IL-6 standard curve (10-2000 pg/mL). In this method serum, plasma or whole blood sources of IL-6 binding to anti-IL-6 may be quantitated calorimetrically.

In clinical practice rheumatoid factor is determined by hemagglutination, latex agglutination, ELISA, the Waaler Rose or sheep-cell agglutination test and nephelometry with variants to these procedures continuing to be developed (Spiritus et al Ann. Rheum. Dis. 63:1169-1171 (2004)). One variant on the Waaler Rose assay involves substituting gelatin particles (Serodia-RA, Fujirebio, Inc. Japan) for red blood cells to improve specificity of serum reactions. Another relatively recent assay combines detection of rheumatoid factor with detection of antibodies to cyclic citrullinated peptide (Kroot Arthritis Rheum. 43:1831-1835 (2000)).

The immunoassays utilized in the methods of the present invention are alternatively sandwich assays employing the antibodies disclosed herein, although other assay formats known in the art may also be used. Additionally, the immunoassays described herein may be adapted to a solid state resin that contains fluorescent tags which allow for quantitation of the amount of a cytokine with fluorescent particle sorting equipment such as that made by Luminex Technologies.

Mouse Model of Inflammation Supporting Arthritis Model: Study of effect of Treatments on LPS-Induced Cytokine Production

Mice (C57-B6 mice, ˜7-8 wks, male, Charles River Labs, 22-28 g) were quarantined and examined daily during 72 hr quarantine period. Any sign of clinical distress, disease, or injury were noted on a daily basis. Mice exhibiting none of these signs were accepted for the study. Accepted mice were transferred to routine maintenance and housed at 5 per cage. Each mouse was identified by an ear tag.

The vehicle or pharmaceutically acceptable carrier for test compounds was 20% hydroxypropyl-beta-cyclodextrin for subcutaneous and interperoneal routes (Groups 6-9). For Groups 1-5, the “Vehicle” as used in this study is 0.1% Tween 80 and 1% carboxymethylcellulose (CMC). The 0.1% Tween 80 and 1% carboxymethylcellulose (CMC) was the Vehicle used in the mice in Groups 1 and 2.

The compounds were administered with pre-formulated material at a dose of 0.2 mL for 25 g mouse (8.0 mL per kg body weight). Final dose was approximately 50 mg/kg of body weight. At the initiation of the study (T=0) animals were weighed and administered with test material or Vehicle. Five 5 mice per treatment per time point group were completed.

At the first time point (T=24 hours) the animals were administered with test material or Vehicle again as above. [0.2 mL for 25 g mouse (8.0 mL per kg body weight)]

| Group # | LPS | Material for dosing | Route | Dose |
|---|---|---|---|---|
| 1 | No | Vehicle | PO | NA |
| 2 | Yes | Vehicle | PO | NA |
| 3 | Yes | SKF86002 | PO | 100 mg/kg |
| 4 | Yes | COMPOUND #2 | PO | 50 mg/kg |
| 5 | Yes | COMPOUND #1 | PO | 50 mg/kg |
| 6 | Yes | COMPOUND #2 | SC | 50 mg/kg |
| 7 | Yes | COMPOUND #1 | SC | 50 mg/kg |
| 8 | Yes | COMPOUND #2 | IP | 50 mg/kg |
| 9 | Yes | COMPOUND #1 | IP | 50 mg/kg |

*COMPOUNDs #1 and #2 correspond to the designations given in Table 1
PO = oral;
SC = subcutaneous;
IP = intraperitoneal.

At the time point T=25 hour, animals were administered 2 mL/kg of an LPS solution (5 mg/mL, lipopolysaccharide from Escherichia coli 0111:B4; dissolved in sterile PBS) or vehicle by intraperitoneal route. At this time point, Group 1 mice received Vehicle only as the non-LPS control and Groups 2 through 9 mice received LPS and Vehicle (Group 2) or Test Materials.

At the time point T=30 hour, mice in Groups 1 through 9 were anesthetized and exsanguinated into EDTA-treated microtainer tubes.

The lungs were observed to assure proper oral dosing had occurred and the blood from each mouse was processed to plasma and divided into two fractions. The plasma samples was stored at −20° C. until testing. Plasma IL-6 was measured by Luminex cytokine multiplex measurements using beads supplied by Linco.

### Results

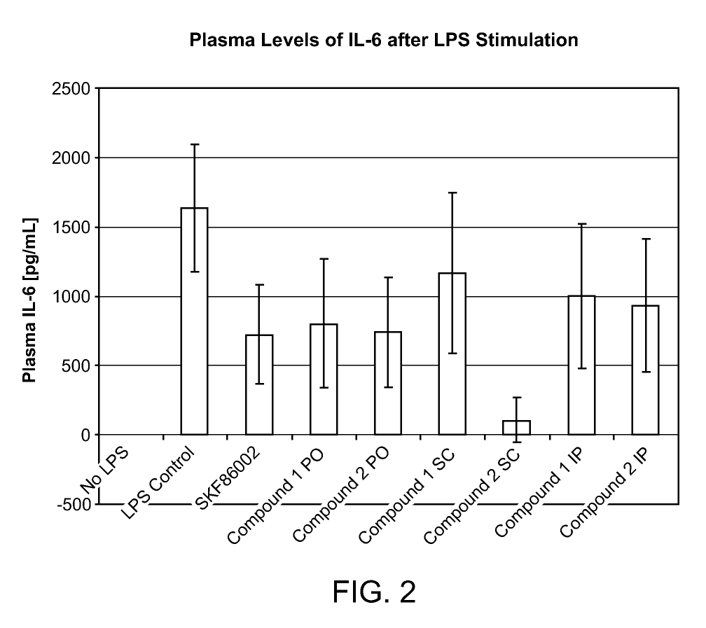

The positive control, SKF86002 inhibited the release of IL-6 in response to LPS by 66% relative to the vehicle control. Similarly COMPOUND #2 and COMPOUND #1 were able to inhibit 51 and 55% of IL-6 release when given orally. Strikingly, while COMPOUND #1 dramatically inhibited IL-6 release to 94% inhibition, COMPOUND #2 inhibit IL-6 release only 51% when administered SC. Results are shown as a bar graph in FIG. 2.

### Collagen Induced Rheumatoid Arthritis (RA) Mouse Model.

Mice were immunized with collagen as follows. Collagen (Bovine Type II, 0.05M in acetic acid) was emulsified with CFA (Complete Freund's Adjuvant) by mixing one volume of CFA with an equal amount of the collagen solution. Mixing was continued until a stable, stiff emulsion resulted. To ascertain the desired stability of the emulsion, 1 drop of emulsion was added into a water-filled beaker. The emulsion was considered stable if it remained in the water as a solid. For the collagen IP boost on day 21, the Bovine Type II collagen (Condrex, Seattle, Wash.) was thawed at 4° C. overnight and then mixed 1:1 with the PBS supplied. 200 μL was injected IP into each mouse.

Four groups of 10 mice (40 total), aged 7-8 weeks were acclimated a minimum of 5 days prior to start of the study. Animals were housed 5 per cage in microisolators in a 12:12 light dark cycle (all work was done in a BioBubble Hood™). Standard maintenance mouse chow diet (Harlan Teklad 8640) was supplied. Water and food were given ad libitum.

### Onset and Severity of Arthritis

High CIA-susceptible DBA/1 mice were utilized which generally develop arthritis 3-5 weeks after immunization with Bovine type TI collagen. Fully developed arthritis, including red and swollen paws, was to be observed 3-5 days after the onset. Active, inflammatory arthritis lasted more than 3 to 4 weeks. Although inflammation eventually subsides, joints generally do not heal and ankylosis will be permanent.

### Experimental Groups: N=10 Mice Per Group

Dosing with test compounds or controls began on Day 20 following initial collagen injection, with one hour prior to the second dose of collagen on Day 21. Time of dosing was approximately the same each day following through the end of the study. The mice groups were fed as follows through the duration of the experiment:

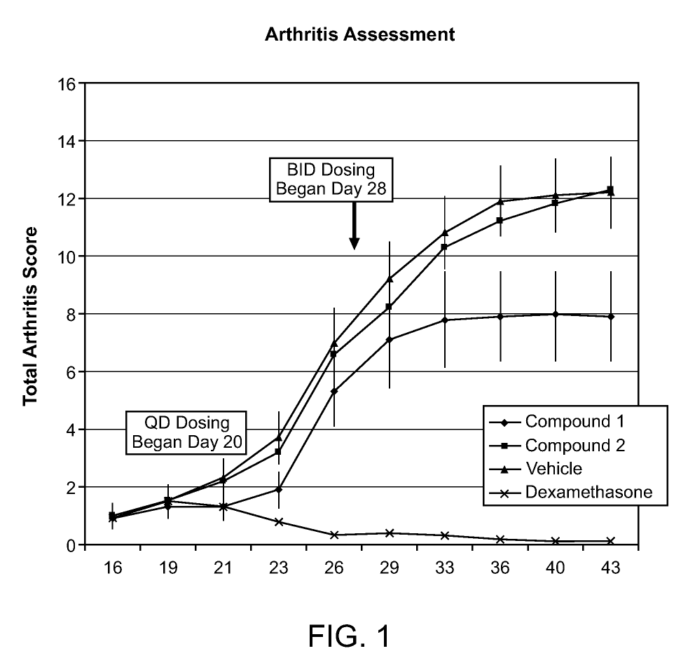

Group 1) Chow (Referred to as “Vehicle” in FIG. 1).

Group 2) Chow (Positive Control-Dexamethasone (DEXA))-Dosed as appropriate.

Group 3) COMPOUND #2 Lot #5 50 mg/kg orally on a daily basis (“PO QD”) from Day 20- to Day 27, then dose increased to twice daily (“BID”) at 50 mg/kg Day 28 to Day 42.

Group 4) COMPOUND #1 Lot #4 50 mg/kg —PO QD from Day 20 to Day 27, then dose was increased to BID at 50 mg/kg Day 28 to Day 42.

COMPOUND #2 and COMPOUND #1 were dosed as suspensions in 0.1% Tween 80, 1% carboxymethylcellulose. The dosing solutions were made on a twice weekly basis and stored at 4° C. between dosing.

Measurements were taken twice weekly from boost and included: body weight measurement and Arthritic Score on a scale of 0 to 4. Arthritic score 0-4 (0=normal; 1=1-2 digit swollen and mildly red; 2=redness and swelling in mid-foot or ankle; 3=>3 paws and ankle joints swollen and very red); 4=all joints swollen and animal is not using the paw.

At onset of arthritis paw measurements were taken with calipers. A baseline of caliper measurements was taken when all visual assessments were zero. Measurements were taken with an electronic caliper that entered measurements directly to an excel spreadsheet.

The study was terminated on Day 42 from initial collagen injection. Cardiac blood was taken from each mouse and plasma prepared in EDTA within 15 min of collection. Plasma was frozen at −20° C. for future analysis of cytokines and eicosatrienoic acids. All left hind paws were removed and stored in formalin for future histology.

### Results

The arthritis score for mice treated with COMPOUND #2 was not significantly different from Vehicle treated animals whether it was dosed once a day or twice a day. In contrast, COMPOUND #1 significantly decreased the arthritic score once it was dosed twice a day (p<0.01). Prior to increasing the dose there was no significant difference from Vehicle. The positive control, dexamethasone, prevented inflammation in the paws in this experiment. All results are illustrated graphically in FIG. 1.

### Pharmacokinetic Study in Mice

All animals were weighed prior to dose administration. Compounds 1 and 2 were suspended in 0.1% Tween 80, 1% carboxymethylcellulose in water for oral gavage. Compounds 1 and 2 were dissolved in 20% hydroxypropyl-beta-cyclodextrin for dosing subcutaneously. The dosing solutions of compounds 1 and 2 were then administered to the mice by oral gavage in Group 1 and subcutaneously in Group 2.

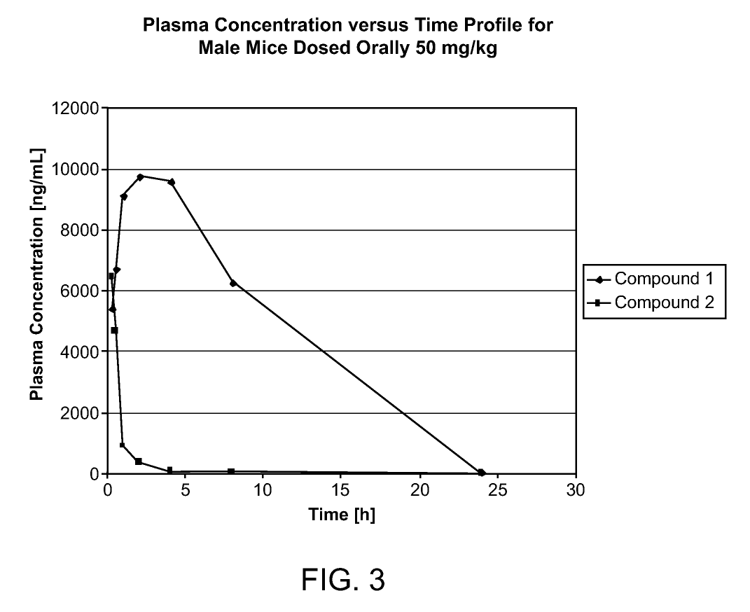

Blood (˜0.5 mL) was collected by cardiac puncture at sacrifice and transferred into vacutainer tubes containing sodium heparin at the time points outlined (See FIG. 3). After collection, the blood samples were centrifuged at approximately 1000×g at 2-8° C. for approximately 15 minutes. Each plasma specimen was collected, aliquoted into 2 microcentrifuge tubes, and stored at about −20° C. until analysis by LC/MS (liquid chromatography/mass spectrometry).

The quantitation method for pharmacokinetics is summarized as follows:

Individual solutions of Compound 1 and 2 at 0.5 mg/mL in DMSO were prepared and stored at −25° C., and used as working standards. To prepare calibration standards for quantitation, the working standard was diluted 1 in 100 into rat plasma to 5 μg/mL (5 μL+495 μL), then diluted further with the same medium by 3-fold serial dilution. A total of seven dilutions were performed yielding eight calibration standards ranging from 2.29 to 5000 ng/mL. Calibration standards 3, 5 and 7 were used as the low, mid and high QC samples, respectively.

Calibration standards, QC samples and study samples were prepared for HPLC injection by precipitating plasma (25 μL) with 3× volumes (75 μL) of ice cold Internal Standard Solution. Following centrifugation at 6100 g for 30 minutes, 40 μL of each supernatant was transferred to a deep-well microplate and diluted with 200 μL of 0.2% formic acid in water. Samples were analyzed using the following LC/MS/MS conditions and equipment parameters:
HPLC: Shimadzu VP System
Mobile Phase: 0.2% formic acid in water (A) and 0.18% formic acid in methanol (B)
Column: 2×10 mm Peeke Scientific DuroGel G C₁₈ guard cartridge
Injection Volume: 100 μl
Gradient: 5% B for 0.5 minutes then 5-95% B in 2 minutes
Flow Rate: 400 μl/min
Mass Spectrometer Applied Biosystems/MDS SCIEX API 3000
Interface: Turbo Spray (ESI) at 400° C.
Ionization Mode Positive Ion
Plasma was analyzed for COMPOUND #2 or COMPOUND #1 concentration by LC/MS as described above.

### Results

Both compounds were detectable in mouse plasma, however, COMPOUND #1 had much greater and longer exposure than COMPOUND #2. The Cₘₐₓ is nearly twice as high for COMPOUND #1 at 9916 ng/mL as compared to 6486 ng/mL for COMPOUND #2. More dramatically, the area under the curve (AUC) is over 20-fold higher for COMPOUND #1 as compared to COMPOUND #2. The mean resident time (MRT) is also markedly higher at 5.6 hours for COMPOUND #1 compared to 1.3 hours for COMPOUND #2. (See FIG. 3) Compounds 1 and 2 have nearly the same IC₅₀ against sEH at 9 and 8 nM, respectively. Since the twice daily dosing was more efficacious than once daily dosing, it is likely that the difference in efficacy in the collagen-induced arthritis model is due to the difference in exposure.

### Cytokine Secretion Assays

It is contemplated that the compounds of the invention reduce autoimmune induced inflammation by reducing autoimmune cytokine levels, wherein the autoimmune induced cytokine is selected from the group consisting of TNF-α, IL-1, IL-6, IL-11, IL-12, IL-17 and IL-23, RANTES, MIP-1α, GMCSF, and MCP-1. The effects of the sEH inhibitor compounds on the secretion of cytokines from cells upon treatment with test compounds and LPS may be evaluated from the following procedure. Frozen human PBMCs are thawed and seeded at 10,000 cells per well of a 96-well plate. The thawed cells are then incubated at 37° C., 5% CO₂ for 15 minutes, followed by incubation with test compounds for 30 minutes prior to induction with LPS (50 pg/mL). After a 24 hour incubation with LPS, the supernatents are collected, centrifuged and frozen at −80° C. The profile of secreted cytokines in culture supernatant are assayed using the 5-plex ImmunoSignal Assay (Luminex). Data from five cytokines (IL-1β, IL-6, MIP-1α, IL-8 and TNF-α) are plotted in a six point curve in duplicate. An Alomar Blue toxicity assay is run and evaluated concurrently.

The vehicle control (N=3) and a 5-plex Luminex assay (LPS panel #2) are performed as described below.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for reducing autoimmune induced inflammation in a subject in need thereof, comprising administering to said subject an effective amount of an autoimmune induced inflammation reducing compound of Formula (I) or a pharmaceutically acceptable salt thereof:
R¹NHC(═O)NHR² (I)
wherein:
Q is O or S; and
R¹ and R² are independently selected from the group consisting of substituted alkyl, aryl, substituted aryl, heteroaryl, substituted heteroraryl, cyloalkyl, substituted cycloalkyl, heterocycloalkyl, and substituted heterocycloalkyl.

2. The method of claim 1 wherein said compound reduces autoimmune induced inflammation by reducing autoimmune cytokine levels, wherein the autoimmune induced cytokine is selected from the group consisting of TNF-α, IL-1, IL-6, IL-11, IL-12, IL-17 and IL-23, RANTES, MIP-1α, GMCSF, and MCP-1.

3. The method of claim 2, wherein the autoimmune induced cytokine levels are reduced by at least 75%.

4. The method of claim 3, wherein the autoimmune induced cytokine levels are reduced by at least 95%.

5. The method of claim 1 wherein said compound reduces autoimmune induced inflammation by increasing the proportion of anti-inflammatory cytokine levels, wherein the anti-inflammatory cytokine is selected from the group consisting of IL-10 and IL-13.

6. The method of claim 5, wherein the increased proportion of anti-inflammatory cytokine levels is greater than about 75%.

7. The method of claim 6, wherein the increased proportion of anti-inflammatory cytokine levels is greater than about 95%.

8. The method of claim 1, wherein said compound reduces autoimmune induced cytokine levels by 75% and increases anti-inflammatory cytokine levels by 75%.

9. The method of any one of claims 1, 2 and 5, wherein the subject in need thereof has been diagnosed with an autoimmune disorder selected from the group consisting of rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis.

10. The method of claim 9 wherein the autoimmune disorder is rheumatoid arthritis.

11. A method for reducing autoimmune induced inflammation in a subject in need thereof, comprising administering to said subject an effective amount of a compound of Formula (II) or a pharmaceutically acceptable salt thereof: 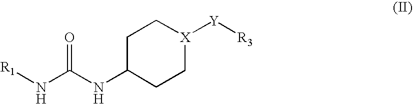
wherein:
R¹ is selected from the group consisting of aryl, substituted aryl, heteroaryl, substituted heteroraryl, cyloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

12. The method of claim 11 wherein said compound reduces autoimmune induced inflammation by reducing autoimmune cytokine levels, wherein the autoimmune induced cytokine is selected from the group consisting of TNF-α, IL-1, IL-6, IL-11, IL-12, IL-17 and IL-23, RANTES, MIP-1α, GMCSF, and MCP-1.

13. The method of claim 12, wherein the autoimmune induced cytokine levels are reduced by at least 75%.

14. The method of claim 13, wherein the autoimmune induced cytokine levels are reduced by at least 95%.

15. The method of claim 11 wherein said compound reduces autoimmune induced inflammation by increasing the proportion of anti-inflammatory cytokine levels, wherein the anti-inflammatory cytokine is selected from the group consisting of IL-10 and IL-13.

16. The method of claim 15, wherein the increased proportion of anti-inflammatory cytokine levels is greater than about 75%.

17. The method of claim 16, wherein the increased proportion of anti-inflammatory cytokine levels is greater than about 95%.

18. The method of claim 11, wherein said compound reduces autoimmune induced cytokine levels by 75% and increases anti-inflammatory cytokine levels by 75%.

19. The method of any one of claims 11, 12, and 15, wherein the subject in need thereof has been diagnosed with an autoimmune disorder selected from the group consisting of rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis.

20. The method of claim 19 wherein the autoimmune disorder is rheumatoid arthritis.

21. The method of claim 11 wherein the compound is selected from the group consisting of 1-[3-(morpholine-4-carbonyl)-phenyl]-3-(4-trifluoromethyl-phenyl)-urea, 1-(1-acetylpiperidn-4-yl)-3-(adamant-1-yl)urea, 1-(1-acetylpiperidyn-4-yl)-3-[4-(trifluoromethyl)phenyl]urea, 1-[1-(methylsulfonyl)piperidin-4-yl]-3-[4-(trifluoromethyl)phenyl]urea and 1-[1-(methylsulfonyl)piperidin-4-yl]-3-(adamant-1-yl)urea.
